(19) United States
(12) Patent Application Publication
(10) Pub. No.: US 2026/0268510 A1
(43) Pub. Date: Sep. 10, 2026

(54) Estimation Of Blood Loss Within A Waste Container Of A Medical Waste Collection System

(71) Applicant: Stryker Corporation, Portage, MI (US)

(72) Inventors: Mayank Kumar, San Jose, CA (US); Jeffrey Scott, Kalamazoo, MI (US); Thang Sy, Sacramento, CA (US); Keng-Tsai Lin, San Jose, CA (US); Steven D. Scherf, Oakland, CA (US); Michael A. Stinchcomb, Spring Lake, MI (US); Alexey Perminov, Belgrade (RS); Nikhil Sharma, Redwood City, CA (US); Benjamin Edinger, Grand Haven, MI (US); Siddarth Satish, Portola Valley, CA (US); Joseph Michael O'Reilly, Kalamazoo, MI (US)

(73) Assignee: Stryker Corporation, Portage, MI (US)

(21) Appl. No.: 18/875,468

(22) PCT Filed: Jun. 16, 2023
(86) PCT No.: PCT/US2023/025603

§ 371 Date: Dec. 16, 2024

Related U.S. Application Data
(60) Provisional application No. 63/353,208, filed on Jun. 17, 2022.

(51) Int. Cl. G06T 7/62; A61B 5/00; A61B 5/02; A61B 5/1455; G06V 10/25; G06V 10/26; G06V 10/764; G06V 20/69

(52) U.S. Cl. CPC G06T 7/62; A61B 5/02042; A61B 5/1455; A61B 5/742; G06V 10/25; G06V 10/26; G06V 10/764; G06V 20/695; G06V 20/698; G06T 2207/10016; G06T 2207/30204

(57) ABSTRACT

Methods for estimating blood loss within a waste container of a medical waste collection system in which an imaging device is supported with a device cradle. The method includes analyzing an image, such as image frames of a video feed, to determine a fluid volume and a blood component within the waste material. The images may be aligned based on reference markers, and the method includes segmenting the images to determine blood, meniscus, and non-blood classes. The fluid meniscus of the waste material may be identified, and mapped to a canonical coordinate space for pixel to volume conversion. Calibration data associated with the reference markers may be used to do so. At least one region of the interest associated with an insert disposed within the waste container is analyzed. The estimated blood loss, based on the determined fluid volume and the blood component, may be displayed in real-time.

### PRIORITY CLAIM

This application claims priority to and all the benefits of U.S. Provisional Patent Application No. 63/353,208, filed Jun. 17, 2022, the entire contents of which are hereby incorporated by reference.

### BACKGROUND

A byproduct of surgical procedures is the generation of liquid, semisolid, and/or solid waste material. The medical waste may include blood, interstitial fluids, mucus, irrigating fluids, and the like. The medical waste may be removed from the surgical site through a suction tube under the influence of a vacuum from a vacuum source to be collected within a waste container.

Estimating blood loss during surgery may be used to monitor intraoperative patient health. Advances in imaging and computing have provided for estimating blood loss by capturing an image of the fluid-containing media, such as a freestanding container. One such system is sold under the tradename Triton by Gauss Surgical, Inc. (Menlo Park, Calif.) and disclosed in commonly-owned U.S. Pat. No. 9,773,320, issued Sep. 26, 2017, the entire contents of which are hereby incorporated by reference. The freestanding container is arranged inline between a suction tube and the vacuum source. Once it is desired to know the quantity of blood within the freestanding container, the user directs a handheld imaging device to capture an image of a region of the container in which an insert is disposed, and manually enter the volume of fluids within the container. While the Triton system has realized significant benefits in estimating blood loss, it would be beneficial for the system to require less involvement from the user, and estimate and display the blood loss in real-time.

The vacuum source may be a facility-integrated vacuum source, or a vacuum source of a medical waste collection system. One exemplary medical waste collection system is sold under the tradename Neptune by Stryker Corporation (Kalamazoo, Mich.), in which at least one waste container is disposed on a mobile chassis. As the Neptune system includes an integrated waste container, it would be further desirable to obviate the need for the freestanding container, which may otherwise become redundant. Doing so, however, may be associated with technical challenges addressed herein. For example, whereas the freestanding container may be disposable after a single use, the waste container(s) of the medical waste collection system arc capital components to be reused over numerous procedures. Soiling of the transparent sidewall of the waste container(s) may affect consistent image quality, and therefore the accuracy of the estimated blood loss. For another example, while the Neptune system includes a subsystem for determining fluid volume within the waste container(s), in certain instances the imaging device may not have access to the volumetric data (e.g., the imaging device and the Neptune system are not in electronic communication). In another instance, the fluid volume determined by the Neptune system may not quantify pre-filled volume, i.e., the volume of fluid in the canister before the start of the procedure. To be sufficiently accurate, image-based determinations of the fluid volume may need to account for camera positioning, frothing of the fluid, and mechanical variances of subcomponents of the medical waste collection system, among other considerations such as lighting or flash variations, glare, and the like.

### SUMMARY

The present disclosure is directed to methods for estimating a blood component within a waste container of a medical waste collection system, preferably in real time as waste material is being drawn into the waste container under the influence of suction. The blood component may be a hemoglobin concentration for estimating blood loss (eBL), and displaying the estimated blood loss (and/or hemoglobin) of the patient. The present disclosure is also directed to devices for performing the methods disclosed herein. The methods may be instructions stored on non-transitory computer-readable medium configured to be executed by one or more processors. The methods may be implemented by a machine-learning (ML) model, trained neural networks, or combinations thereof. The neural network may be trained on image datasets of any number of factors, or combinations thereof, including but not limited to the location of a camera relative to the waste container, location of a flash reflected on a surface of the waste container, color component values of one or more regions of interest (i.e., imaging region(s) of the waste material disposed between an insert and an inner surface of the waste container), color component values of at least two reference markers of a known color profile and affixed to an outer surface of the waste container, phase characteristics of the waste material (e.g., blood, non-blood, fluid meniscus, froth, hemolysis levels, etc.), or the like. The image datasets may be extensive and diverse to train the neural network on at least nearly an entirety of expected scenarios within the expected parameters of the medical waste collection system. The image datascts may also be outside the expected parameters, such as one, two, or three or more standard deviations therefrom. Therefore, in operation, the image or image frames a video feed being analyzed by the neural network on one or more processors is capable of estimating, in real-time, blood loss within the waste material, particularly as the waste material is being drawn into the waste container under the influence of a vacuum, often in a turbulent and unpredictable manner.

An imaging device including the camera is configured to capture images of the waste container. The captured image may be analyzed by the one or more processors, or image frames of a video feed being captured by the camera may be analyzed by the processor(s). As a result, the eBL may be continuously updated and streamed on a display. A device cradle may be removably coupled or rigidly secured to a chassis of the medical waste collection system. The device cradle may include a shield, and at least one arm extending from the shield. One of the arms may include a hinge. A latch or other suitable locking mechanism may be coupled to the shield. An aperture provides communication between rear and front recesses. The aperture is defined at a position within the rear recess so as to position the camera and a light source at the precise location relative to the waste container. The front recess is sized to receive and support the imaging device.

A method of estimating the volume of blood within the waste container may include operating in a standby or low-power mode. The low-power mode may include capturing, with the camera, image frames of a video feed with the flash off or at a lower level, and at a lower frame rate. The image frames may be preprocessed, and an activity recognition algorithm may analyze the image frames to detect a pixel-based location of the fluid meniscus in each of the images. The activity recognition algorithm determines whether the pixel-based location of the fluid meniscus changes by a greater amount than a predetermined threshold in a predetermined period of time. The activity recognition algorithm may compare the pixel-based location the fluid meniscus between successive image frames of the video feed, and compare the changes against the predetermined threshold. If the changes remain below the predetermined threshold, the system is maintained in the low-power mode. If the change in the fluid meniscus is above the predetermined threshold, the method may include activating or increasing a level of the light source the imaging device, and increasing the feed rate of the video feed.

In a first variant, the method may include capturing one or more single or multi-exposure photos for the subsequent processing and analysis. In another variant, the method may include analyzing at least one of the image frames of the video feed, and preferably multiple image frames in a series according to the algorithm. The step may be performed while the waste material is being drawn into the waste container (e.g., the vacuum source is activated), or with the vacuum deactivated, or combinations thereof.

The method includes detecting at least one reference marker(s), for example, quick response (QR) code(s). The reference markers may be affixed to an outer surface of the waste container. The QR code(s) may be associated, in memory, with calibration-based data associated with the waste container. The camera may be activated in a calibration mode, and at least one calibration run is performed in which at least one known fluid volume is suctioning into the waste container. The processor performs the image-based volumetric determination to be described, including ascertaining a vertical, y-axis value of the fluid meniscus, and associates each of the y-axis value and the known fluid volume(s) with the unique code of the QR codes. The calibration data is stored on memory.

The method includes the step of aligning the image. The step of aligning the image may be based on the step of detecting the reference markers. The processor is configured to detect the QR codes and rotate the image accordingly. Additionally or alternatively, the camera may be configured to detect a landmark or fiducial of the waste container or the chassis, and align the image accordingly. The image frames are segmented, and a center of mass of the waste container may be determined. The camera may be calibrated, and a center of optical sensor may be determined. The camera calibration may be performed by determining an orientation of the imaging device. The image coordinates may be mapped to canonical coordinates. One or more of the alignment blocks of the QR may be used for the transformation to two-dimensional canonical coordinates, then to three-dimensional canonical coordinates. The cropped image frames in the canonical coordinate space may include the at least two reference markers, and a portion of the waste container disposed therebetween. A width of the cropped and mapped image frames is approximately equal to a width of the at least two reference markers. The cropping of the adjusted images is optional. The pixel point of the center of mass and the pixel point of the center of the optical sensor may each be converted to a three-dimensional point in the canonical space of the waste container. From the orientations of the waste container and the imaging device, mathematical corrections are determined. The corrected volume is determined based on the image-based volume determination and the mathematical corrections.

The captured image or image frames of the video feed may be segmented using the neural network, including the waste material contained therein. The neural network may characterize whether a pixel is of blood, fluid meniscus, non-blood, and optionally froth, foam, and/or a fluid surface. Each pixel is assigned a value from the neural network, and then assigned a class label based on the value. A location of the fluid meniscus is determined based on the segmentation mask. The step may include fitting a parabola to pixels having the class label of meniscus. In instances where the fluid meniscus is below the camera, the parabola includes a lower vertex and opens upwardly. If the fluid meniscus is above the camera, the parabola includes a higher vertex and opens downwardly. The location of the fluid meniscus is the y-axis minimum or maximum value of the vertex of the parabolic curve. Any other polynomial function can be used to approximate the meniscus line, and accordingly minimum or maximum point can be found to determine the location of the fluid.

The method then includes the step of pixel-to-volume mapping to determine the fluid volume within the waste container. The step may include retrieving or receiving the calibration data from the memory, and the image-based volumetric determination is the y-axis value of the fluid meniscus, in the canonical coordinates, as adjusted by the calibration data.

In certain implementations, the method includes extracting a region of interest (ROI) from the images. The region of interest may be first and second imaging surfaces of the insert. The region of the image associated with the imaging feature of the insert to quantify a concentration the blood component in the waste material. Further optional steps may include extracting palette colors from the reference marker(s), noise removal, extracting features (e.g., red-green-blue (RGB) values from the insert), and performing light normalization to account for light intensity variations. The light normalization may include neural network being trained to predict the color profile of a lower one of the reference markers based on the color profiles of an upper one or more of the reference markers, and applying color correction based on the predicted color profile of the lower reference marker. Alternatively, certain aspects of the method are consolidated to be analyzed by the neural network being trained on image datasets of such aspects.

The estimated blood loss may be displayed on one or more displays, for example, the touchscreen display of the imaging device. The eBL may be displayed and updated at desired intervals or in real-time. In particular, in the video mode the camera may take the video feed in which each of the blood component and the fluid volume is repeatedly determined in a near-instantaneous manner. The touchscreen display of the camera may show the field of view of the camera, and further may be augmented with information of use to the user.

Therefore, according to an aspect of the present disclosure, a method of estimating blood loss of waste material within a waste container of a medical waste collection system is provided. An imaging device captures a video feed of the waste container and the waste material disposed therein as the vacuum source of the medical waste collection system is drawing the waste material into the waste container. One or more processors analyze image frames of the video feed to determine the volume of the waste material within the waste container. The one or more processors analyze the image frames to determine a concentration of the blood component within the waste material. The one or more processors estimate the blood loss based on the determined volume and the determined concentration of the blood component. And the estimated blood loss is displayed on a display.

According to another aspect of the present disclosure, a method of estimating blood loss of waste material within a waste container of a medical waste collection system is provided. An imaging device captures a video feed of the waste container and the waste material disposed therein as the vacuum source of the medical waste collection system is drawing the waste material into the waste container. Image frames of the video feed are analyzed by one or more processors to determine a concentration of a blood component within the waste material. The analysis of the image frames is facilitated by a neural network trained on image datasets of at least one of relative positioning of a camera of the imaging device relative to the waste container, position of a flash reflected on a surface of the waste container, color component values associated with at least two reference markers affixed to the surface of the waste container, and color component values associated with an imaging surface of an insert disposed within the waste container. The blood loss is estimated based on a volume and the determined concentration of the blood component, and displayed on a display.

In certain implementations, the neural network is further trained on the image datasets of regions of interest associated with at least two imaging surfaces of the insert with each of the at least two imaging surfaces providing a different color component value to the waste material disposed between the insert and the waste container. Alternatively, the neural network is further trained on the image datasets of a region of interest associated with the imaging surface of the insert providing a color gradient to the waste material disposed between the insert and the waste container. The neural network may be further trained on the image datasets of at least one of various volumes of the waste material and differing phase characteristics of the waste material. The image frames may be mapped from an image coordinate space to a canonical coordinate space, and provided to the neural network.

According to still another aspect of the present disclosure, a method of estimating blood loss of waste material within a waste container of a medical waste collection system is provided. An image of the waste container and waste material disposed therein is captured by the imaging device. One or more processors are configured to process the image, which includes: processing, with one or more processors, the image, wherein the step of processing the image further comprises: determining a location of a fluid meniscus; aligning the image; mapping the image from an image coordinate space to a canonical coordinate space; and converting a y-axis value of the mapped image in the canonical coordinate to a determined volume. The method includes detecting, with one or more processors, at least two reference markers affixed to the waste container, wherein the reference marker includes location data. A region of interest in a raw image is extracted based on the location data. The image is analyzed to determine a concentration of a blood component within the waste material. The blood loss is estimated with the one or more processors based on the determined volume and the determined concentration of the blood component. The estimated blood loss is displayed on the display.

In certain implementations, the imaging device captures the video feed at a first frame rate. The one or more processors determine a rate at which the volume of the waste material is increasing within the waste container. The one or more processors may detect a pixel-based location of a fluid meniscus in the image frames, and determine whether the pixel-based location of the fluid meniscus changes by an amount greater than a predetermined threshold in the predetermined period of time. The image frames may be down sampled prior to the step of detecting the pixel-based location of the fluid meniscus. The imaging device is configured to capture the video feed at a second frame rate greater than the first frame rate if the rate greater than a predetermined threshold. The flash may be activated, or its level increased, if the rate is greater than the predetermined threshold. This approach aids in reducing the power requirements for running the device continuously.

In certain implementations, the one or more processors, may detect at least two reference markers affixed to the waste container. The one or more processors may align the image or image frames based on relative positioning of the at least two reference markers. The least two reference markers are QR codes arranged in a generally vertical configuration, wherein the step of aligning the image frames further comprises aligning the QR codes to be exactly vertical.

The present disclosure also provides for a method of correcting for tilt of waste container of a medical waste collection system, and/or tilt or the imaging device supported in a device cradle. An image or video feed of the waste container and waste material disposed therein is captured by the imaging device. One or more processors receive the image frames. The one or more processors detect locations at least two reference markers within the image frame, and determine an orientation of the waste container based on the locations of the locations at least two reference markers. An orientation of the imaging device is determined, for example based on internal gyroscopes, and the one or more processors determine mathematical corrections based on the orientation of the waste container and the orientation of the imaging device. The mathematical corrections are applied to determine a corrected volume of the waste material disposed within the waste container.

The at least two reference markers may be AruCo codes, and the image alignment may compensate for tilt of the waste container by determining an orientation of the AruCo codes. The AruCo codes may be disposed on an alignment frame affixed to the waste container. In certain implementations, the image frames are analyzed to determine a concentration of a blood component within the waste material. The one or more processors estimate blood loss based on the corrected volume and the determined concentration of the blood component. The estimated blood loss may be displayed on a display.

In certain implementations, the one or more processors may segment the image or the image frames by characterizing whether pixel values of each of the image frames are blood, meniscus, non-blood, froth of a fluid surface, or other phase characteristic of the waste material. Class labels are assigned class labels based on the pixel values. A location of the fluid meniscus may be determined based on the class label of meniscus. The one or more processors may fit a parabola to the pixels having the class label of meniscus, wherein the step of determining the location of the fluid meniscus further comprises determining a y-axis value of a maximum or a minimum of the parabola. The fluid meniscus may be below a level of the imaging device such that the parabola opens upwardly, and the location of the fluid meniscus may be a y-axis value of a minimum of the parabola. The fluid meniscus may be above the level of the imaging device such that the parabola opens downwardly, and the location of the fluid meniscus may be the y-axis value of a maximum of the parabola. The one or more processors may receive container-specific calibration data, and map the y-axis value to a datum. The one or more processors may convert the y-axis value to a volumetric value.

In certain implementations, the image or the image frames may be mapped from an image coordinate space to a canonical coordinate space. Homography of localization markers (e.g., four localization or reference markers) may be generated and the image coordinates may be transformed to two-dimensional canonical coordinates. The localization markers and the reference markers may be the same or different. The two-dimensional canonical coordinates may be transformed to three-dimensional canonical coordinates.

According to another aspect of the present disclosure, a method of calibrating for mechanical variances in a waste container of a medical waste collection system includes an imaging device is supported in a device cradle. The imaging device captures images of the waste container and the waste material disposed therein in a calibration mode in which the vacuum source of the medical waste collection system draws at least one known volume the waste material into the waste container. The one or more processors analyze the image frames of the video feed to determine y-axis values of a fluid meniscus of the waste material within the waste container. Calibration data is stored on memory with the data associating the at least one known value to the y-axis values of the fluid meniscus.

In certain implementations, a reference marker affixed to the waste container may be detected by the one or more processors. The reference marker includes optically-readable data or code. The one or more processors associate the calibration data with the optically-readable data of the reference marker. The image frames of the video feed may be analyzed by the one or more processors to determine the volume of the waste material within the waste container. The volume is displayed on a display, and the user interface is configured to receive a confirmatory input of the volume of the waste material.

According to still another aspect of the present disclosure, a method of estimating blood loss of waste material within a waste container of a medical waste collection system includes an imaging device is supported in a device cradle. The imaging device captures a video feed of the waste container and the waste material disposed therein as the vacuum source of the medical waste collection system is drawing the waste material into the waste container. The one or more processors segment the image frames of the video feed by characterizing whether pixel values of each of the image frames are blood, meniscus, or non-blood, and optionally, froth or a fluid surface. The one or more processors assign class labels to the pixels based on the pixel values, and determine a location of the fluid meniscus based on the class label of meniscus. The image frames are mapped by the one or more processors from an image coordinate space to a canonical coordinate space. The one or more processors determine the volume of the waste material within the waste container based on the location of the fluid meniscus.

In certain implementations, the one or more processors detect at least two reference markers affixed to the waste containers. The mapped image frames are cropped by the one or more processors. The cropped, mapped image frames may be in the canonical coordinate space and include the at least two reference markers, and a portion of the waste container disposed therebetween. A width of the cropped and mapped image frames may be approximately equal to a width of the at least two reference markers. The one or more processors may align the image frames based on relative positioning of the at least two reference markers.

According to still yet another aspect of the present disclosure, a device cradle for supporting an imaging device for capturing images of a waste container of a medical waste collection system is provided. The device cradle includes a shield coupled to a front casing. The shield defines a rear recess configured to cover the window, a front recess, and an aperture providing fluid communication between the rear recess and the front recess. The front recess is sized to receive the imaging device and the aperture positioned relative to the front recess to be aligned with a camera and a flash of the imaging device.

In certain implementations, at least one arm coupling the shield to the front casing. The device cradle includes a hinge coupling the at least one arm and the shield and configured to permit the shield, with the imaging device disposed therein, to be pivoted to a configuration in which the window is viewable. A front housing may be movably coupled to the shield. At least nearly an entirety of the waste container is con figured to be viewable in a field of view of the camera of the imaging device.

### BRIEF DESCRIPTION OF THE DRAWINGS

FIG. 1 shows a medical waste collection system configured to suction medical waste through a suction tube and a manifold to be collected in a waste container. A device cradle is coupled to a chassis of the medical waste collection system and positioned to removably receive an imaging device configured to capture an image of the waste container.

FIG. 2 is a perspective view of the device cradle in a first configuration.

FIG. 3 is a perspective view of the device cradle in a second configuration.

FIG. 4 is an elevation view of another implementation of the device cradle.

FIG. 5 is a plan view of the waste container with a schematic representation of a field of view of the imaging device.

FIG. 6 is an elevation view of the waste container with a schematic representation of a field of view of the imaging device.

FIG. 7 is a representation of the imaging device capturing a video feed of the waste container while supported within the device cradle.

FIG. 8 is a perspective view of the waste container including an insert disposed therein. An alignment frame and reference markers are coupled to the exterior of the waste container.

FIG. 9A is a method in which one or more images are captured and analyzed by one or more processors for estimating blood loss.

FIG. 9B is a method in which image frames of a video feed are analyzed by one or more processors for estimating blood loss.

FIG. 10 shows the waste container with fluids disposed therein with machine vision output overlying the reference markers for alignment of the image(s).

FIG. 11 shows the waste container with the processor identifying the marker and an external structure of the chassis of the medical waste collection system for alignment of the image(s).

FIGS. 12A-12C are representative of the processing using internal features of the waste container for alignment of the image(s).

FIG. 13 is a method of compensating for tilt of the waste container.

FIGS. 14A and 14B show alternative implementations of the reference markers for compensating for tilt of the waste container.

FIGS. 15A-15F are representations of certain steps of the method applied to a first image frame being processed during collection of the waste material in the waste container.

FIGS. 16A-16F are representations of certain steps of the method applied to a second image frame being processed during collection of the waste material in the waste container.

FIGS. 17A-17F are representations of certain steps of the method applied to a third image frame being processed during collection of the waste material in the waste container.

FIGS. 18A-18F are representations of certain steps of the method applied to a fourth image frame being processed during collection of the waste material in the waste container.

FIG. 19 is a partial view of the waste container depicting the machine vision output overlying the reference marker, and machine vision output associated with a region of interest.

FIG. 20 is another implementation of the method for estimating blood loss in which the method may be executed in a machine learning environment.

### DETAILED DESCRIPTION

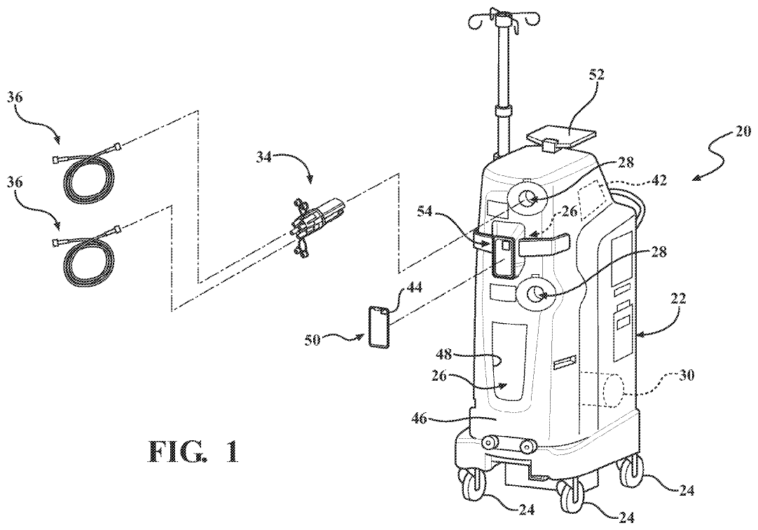

FIG. 1 shows a medical waste collection system 20 for collecting waste material generated during medical procedures. The medical waste collection system 20 includes a chassis 22, and wheels 24 for moving the chassis 22 within a medical facility. At least one waste container 26 is supported on the chassis 22 and defines a waste volume for receiving and collecting the waste material. In implementations in which there is more than one waste container, an upper waste container 26 may be positioned above a lower waste container 26, and a valve (not shown) may be facilitate transferring the waste material from the upper waste container 26 to the lower waste container 26. A vacuum source 30 is supported on the chassis 22 and configured to draw suction on the waste container(s) 26 through one or more internal lines. The vacuum source 30 may include a vacuum pump, and a vacuum regulator configured to regulate a level of the suction drawn on the waste container(s). Suitable construction and operation of several subsystems of the medical waste collection system 20 are disclosed in commonly-owned U.S. Pat. No. 7,621,989, issued Nov. 24, 2009, U.S. Pat. No. 10,105,470, issued Oct. 23, 2018, and U.S. Pat. No. 11,160,909, issued Nov. 2, 2021, the entire contents of which are hereby incorporated by reference. Subsequent discussion is with reference to the upper waste container, but it should be appreciated that the objects of the present disclosure may be alternatively or concurrently extended to the lower waste container.

The medical waste collection system 20 includes at least one receiver 28 supported on the chassis 22. The receiver 28 defines an opening sized to removably receive at least a portion of a manifold 34. A suction path may be established from a suction tube 36 to the waste container 26 through the manifold 34 removably inserted into the receiver 28. In other words, the vacuum generated by the vacuum source 30 is drawn on the suction tubes 36, and the waste material is drawn from the surgical site through the suction tube 36, the manifold 34, and the receiver 28 to be collected in the waste container 26. The manifold 32 may be a disposable component with exemplary implementations of the receiver 28 and the manifold 32 disclosed in commonly-owned U.S. Pat. No. 10,471,188, issued Nov. 12, 2019, the entire contents of which are hereby incorporated by reference.

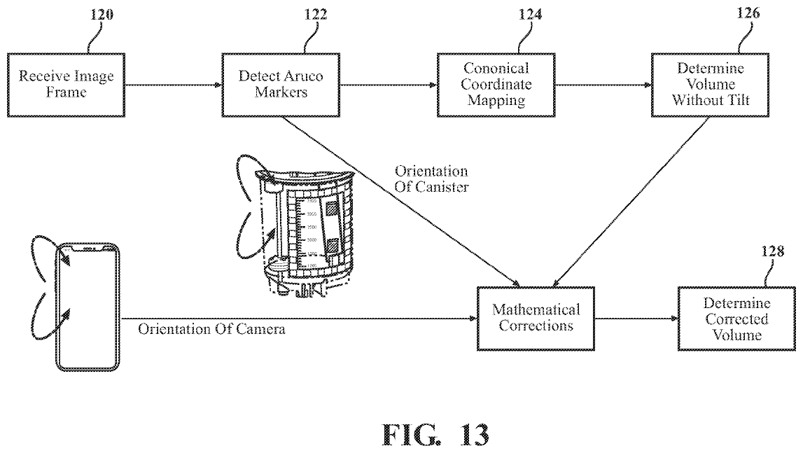

The medical waste collection system 20 includes a fluid measuring subsystem 38, a cleaning subsystem 40, and a container lamp or backlight. An exemplary implementation of the fluid measuring subsystem 38 is disclosed in the aforementioned U.S. Pat. No. 7,621,898 in which a float element 79 is movably disposed along a sensor rod 80 (see also FIG. 13). Based on signals received from the fluid measuring subsystem 38 indicative of a fluid level, a controller 42 in electronic communication with the fluid measuring system 38 is configured to determine a fluid volume of the waste material in the waste container 26. The cleaning subsystem 40 may include sprayers rotatably disposed within the waste container 26 and configured to direct pressurized liquid against an inner surface of the waste container 26, as disclosed in the aforementioned U.S. Pat. No. 10,105,470. Lastly, the container backlight is configured to illuminate an interior of the waste container 26. The container backlight may be activated based on an input to a user interface 52, or another device in communication with the controller 42.

The chassis 22 includes a front casing 46 that defines at least one cutout or window 48 to expose a portion of the waste container 26. The waste container 26 may be formed with transparent material through which a user may visually observe the waste material collected within the waste containers 26, and, if needed, visually approximate a volume of the waste material collected therein with volumetric markings disposed on an outer surface of the waste container 26 (see FIG. 13). The waste container 26 being optically clear also permits the waste material collected therein to be imaged by a camera 50. The camera 50 is also referred to herein as an imaging device in which the imaging device includes the camera 50, a user interface (e.g., a touchscreen display), and optionally a flash or light source. The images from the camera 50 may be transmitted to and processed by one or more processors 44, hereinafter referred to in the singular, to determine a blood component within the waste material. The blood component may be a blood concentration within the waste material (e.g., hemoglobin concentration). More particularly, optical properties of the waste material may be analyzed and processed to determine the blood component as disclosed in commonly-owned U.S. Pat. No. 8,792,693, issued Jul. 29, 2014, the entire contents of which are hereby incorporated by reference. The blood volume within the waste material (i.e., patent blood loss) may be estimated from the determined blood component and the volume of the waste material.

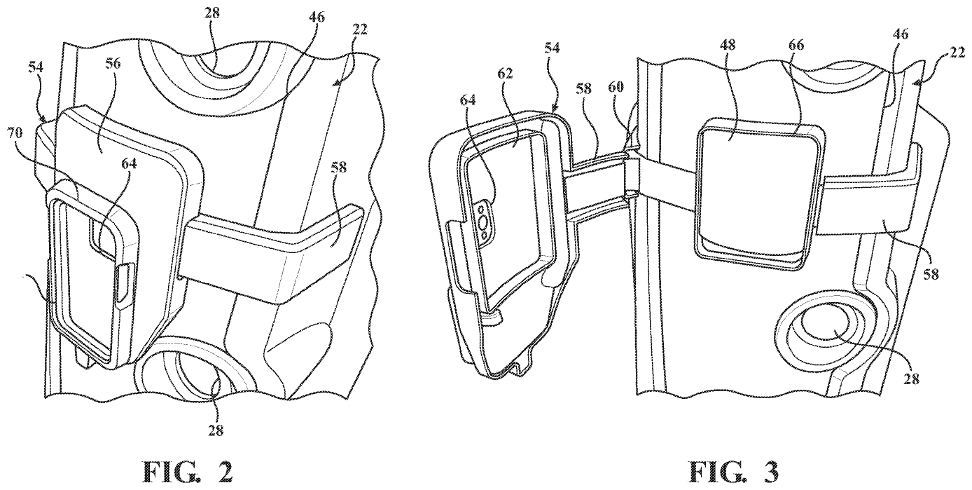

With further reference to FIGS. 2 and 3, a device cradle 54 is removably coupled or rigidly secured to the chassis 22. The device cradle 54 positions the camera 50 relative to the waste container 26 in a precise manner to provide for continuous image capture (e.g., a video feed) of at least nearly an entirety of the waste container 26. The video feed results in continuous data from which the fluid volume, and the blood component therein, may be determined in real-time. As a result, the estimated blood loss (eBL) may be continuously updated and streamed on a display (e.g., the touchscreen display of the imaging device 50, a user interface 52 of the medical waste collection system 20, and/or another display terminal). Such advantages are not readily feasible with a handheld imaging device that requires the user to manually support the camera during image capture, and to manually input an estimated volume based on a visual observation. Moreover, the image-based volumetric determinations obviate the need for the imaging device 50 be in data communication with the controller 42 of the medical waste collection system 20. Still further, the precise positioning may be designed to reduce glare and eliminate variations or aberrations in lighting to improve accuracy of the algorithmic determinations.

The device cradle 54 may be removably coupled or rigidly secured to the front casing 46 or another suitable surface or internal structure of the chassis 22. For example, complementary couplers (e.g., flange-in-slot, detents, etc.) may facilitate the removable coupling of the device cradle 54 with the chassis 22. The device cradle 54 may include a shield 56 configured to be disposed over the window 48 defined by the front casing 46. At least one arm 58 may extend from the shield 56 and be coupled to the chassis 22. In certain implementations, one of the arms 58 may include a hinge 60 configured to permit the device cradle 54 to be moved between first and second configurations in which the shield 56 covers and does not cover the window 48, respectively. For example, in certain procedures it may be desired to forego utilization of eBL, and/or to otherwise view the contents of the waste container 26. A latch or other suitable locking mechanism coupled to the shield 56 may be actuated, and the shield 56 may be pivoted about the hinge 60 to the second configuration, as shown in FIG. 3. For a subsequent procedure or as otherwise desired, the user may pivot the shield 56 about the hinge 60 to return the device cradle 54 to the first configuration.

The device cradle 54 may include a rear recess 62, a front recess 66, and an aperture 64 providing communication between the rear and front recesses 62, 66. The rear recess 62 may be sized and shaped to mate with a rim or lip disposed within the window 48 of the front casing 46 to limit or prevent ingress of ambient light. Alternatively, a resilient flange may extend from a rear of the shield 56 to engage the front casing 46 to do so. The aperture 64 is defined at a position within the rear recess 62 so as to position the camera 50 and a light source (e.g., a flash of the imaging device) at the precise location relative to the waste container 26 in a manner to be further described. The front recess 68 is sized to receive and support the imaging device 50. The dimensions of the front recess 68 may vary based on types of the imaging device 50 itself. In other words, the imaging device 50 may be a smartphone, a tablet, or a custom-designed device, and variants of the device cradle 54 may be compatible with specific models of the device (e.g., Apple iPhone®, Samsung Galaxy®, Google Pixel®, etc.). The illustrated implementation of the device cradle 54 may be for use with the Apple iPhone 13 ProMax® operating an iOS® operating system. In such an arrangement, the device cradle 54 is dimensioned such that the camera and the flash-located at an upper corner of the Apple iphone 13. ProMax®—is in the precise position relative to the waste container 26. In such an arrangement, the front recess 68 is positioned lower than the rear recess 62. Further, the front recess 68 may be sized to support the imaging device 50 in a protective case. In certain implementations, a front housing 70 may be movably coupled to the shield 56 with a second hinge and latching mechanism (not shown) pivotably coupling the front housing 70 to the shield 56.

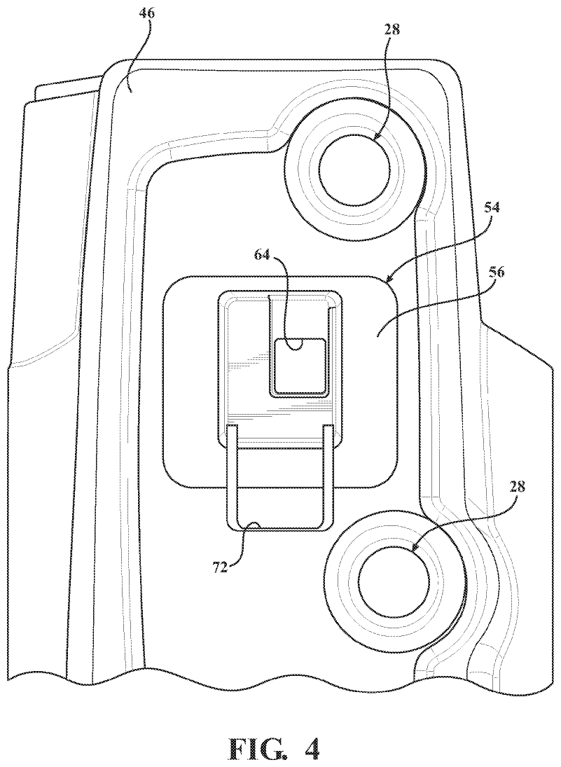

FIG. 4 shows another implementation of the device cradle 54 in which geometries associated with the window 48 of the front casing 46 supports the device cradle 54. For example, small gaps or slots may be present between the front casing 46 and the outer surface of the waste container 26 about the perimeter of the window 48. Certain components of the device cradle 54 may be integrated with the waste container 26 for the device cradle 54 to be placed inside the front casing 46 and external to the waste container 26. The aperture 64 is sized and positioncd to be aligned with the camera 50 and the flash. A lip 72 of the device cradle 54 may be sized and shaped to receive a base of the camera 50 and position the camera 50 against the shield 56 with the camera and flash aligned with the aperture 64. Other means of supporting the camera 50 are contemplated, such as magnets, clips, straps, and the like. The device cradle 54 may be positioned on the chassis 22 to avoid obscuring regulatory labeling or other components of the medical waste collection system 20 requiring accessibility.

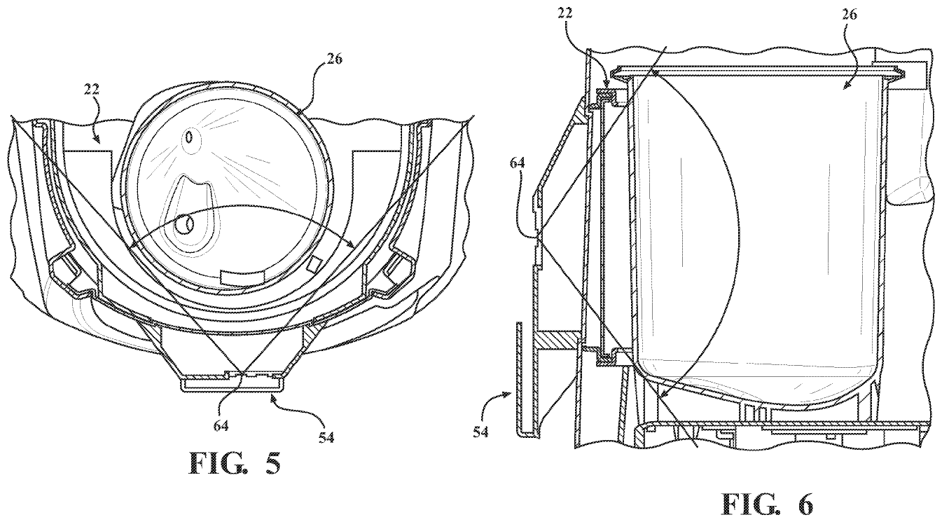

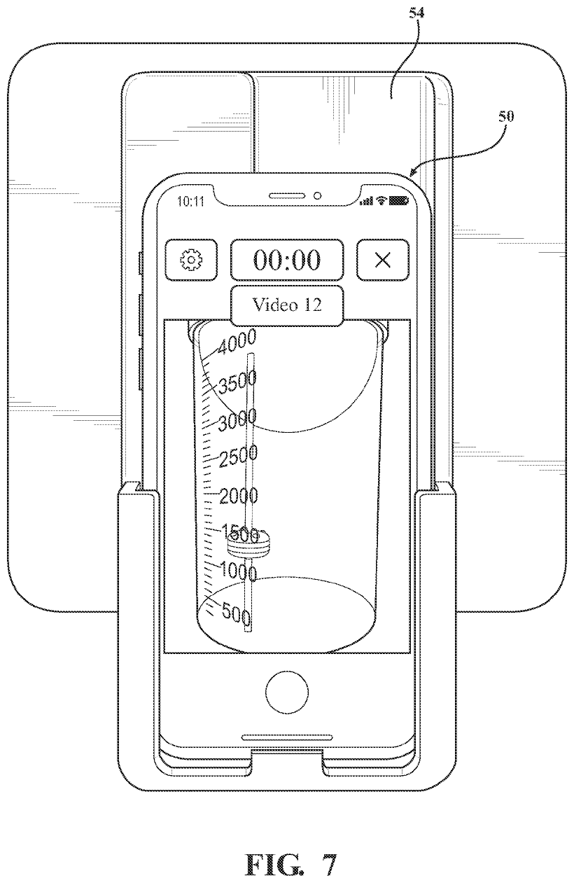

As mentioned, the device cradle 54 supports the camera 50 for the waste container 26 to be within a field of view of the camera. In certain implementations, an entirety of the waste container 26 is within the field of view. Referring now to FIGS. 5 and 6, a representation of the device cradle 54 is shown with the aperture 64 identified to approximate the position of the camera 50. The device cradle 54 may be dimensioned such that the field of view of the camera 50 spans an entire width of the waste container 26, and optionally, to provide desired spacing between the flash of the imaging device 50 and the surface of the waste container 26. The designed spacing may be tuned to reduce glare while adequately illuminating the waste container 26 with the flash of the imaging device 50. Further, the field of view of the camera 50 may span from below a bottom of the waste container 26, to above a top of the waste container 26 or a predetermined fill level thereof. FIG. 6 shows the field of view of the camera 50 including up to approximately 4,000 mL of fluid within the waste container 26. The distance by which the camera 50 is spaced apart from the surface of the waste container 26 may be based on the aspect ratio of the camera 50 (e.g., 4:3, 16:9, etc.). In one example, the precise positioning includes the camera 50 being spaced apart from the surface of the waste container 26 by a distance within the range of approximately 30 millimeters (mm) to 70 mm, and more particularly within the range of approximately 40 to 60 mm, and even more particularly at approximately 50 mm. In certain examples, a horizontal viewing angle may be within the range of approximately 80° to 90°, and a vertical viewing angle may be within the range of approximately 100° to 105°-corresponding lens viewing angles may be within the range of approximately 110° to 120°. Additional optical components such as a fish-eye lens and mirrors may be used on the device cradle 54 to expand the field of view of the camera 50. With the camera and the flash facing towards the waste container 26, the touchscreen display of the imaging device 50 remains visible and operable, including displaying the field of view or an augmented field of the camera, as shown in FIG. 7. It is further appreciated that the arrangement facilitates the camera 50 being removable from and replaceable within the device cradle 54 in a quick and intuitive manner.

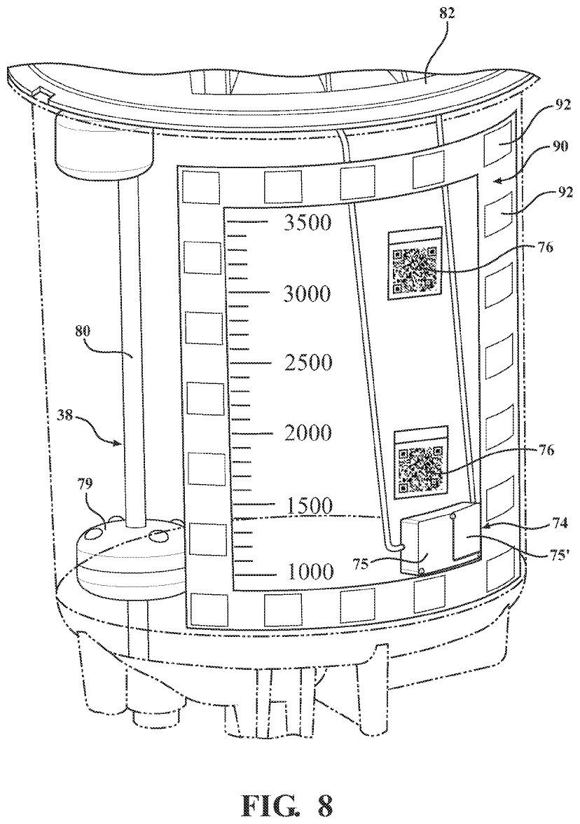

Referring now to FIG. 8, an insert 74 is disposed within the waste container 26 to be within the field of view of the camera 50. The insert 74 includes several geometries, at least one of which is an imaging surface 75 to be spaced apart from an inner surface of the waste container 26 to define a gap of known and fixed distance. The gap permits a thin layer of fluid to be situated between the insert 74 and the inner surface of the waste container 26 that exhibits a region of at least substantially uniform color that is below a color intensity to cause signal saturation. An exemplary implementation the insert 74 is disclosed in the aforementioned U.S. Pat. No. 9,773,320, and commonly-owned International Patent Application No. PCT/US2013/015437, filed Mar. 17, 2023, the entire contents of which are hereby incorporated by reference. The insert 74 may be mounted to a container lid 82 of the waste container 26.

The imaging surface 75 may include first and second imaging surfaces 75a, 75b positioned lateral to one another in a side-by-side arrangement (i.e., a multi-level insert). With standoffs of the insert 74 directly contacting the inner surface of the waste canister 26, the first imaging surface 75a is spaced apart from the inner surface by a first distance and the second imaging surface 75b is spaced apart from the inner surface by a second distance greater than the first distance. In one example, the first distance is 1.7 millimeters, and the second distance is 2.2 millimeters. It is more broadly contemplated that the first distance may be within the range of approximately 0.7 to 5.7 millimeters, and more particularly within the range of 1.2 to 3.7 millimeters, and the second distance may be within the range of approximately 1.2 to 6.2 millimeters, and more particularly within the range of 1.7 to 4.2 millimeters. The first imaging surface 75a and the second imaging surface 75b may be separated by a ridge having a thickness equal to a difference between the first distance and the second distance. For example, the ridge may be approximately 0.5 millimeters. It should be appreciated that the insert 74 may include two, three, four, or five or more imaging surfaces with the illustrated implementations being non-limiting examples. Alternatively, the imaging surface 75 may also include a continuous gradient imaging surface such as in the shape of a wedge to allow several levels of increasing fluid color intensity being measured.

The side-by-side arrangement, among other advantages, provides for improved cleanability of the insert 74. The sprayers of the cleaning subsystem arc rotatably coupled to the container lid 82 of the waste canister 26, and the sprayers direct the liquid downwardly and radially outwardly (schematically represented by arrows) towards the inner surface. The rotatable sprayers of the cleaning subsystem direct pressurized fluid towards the insert 74. The relative spacing of the first and second imaging surfaces 75a, 75b may be based on a direction of rotation of the sprayers of the cleaning subsystem. More particularly, the first and second imaging surfaces 75a, 75b may be arranged so that the pressurized liquid contacts the first imaging surface 75a (i.e., closer to the inner surface) prior to contacting the second imaging surface 75b. Should any semi-solid or solid debris be situated between the insert 74 and the inner surface, the aforementioned flow direction increases the likelihood of dislodging the debris. Therefore, the thin layer of medical waste is better flushed from the gap between the imaging feature 75 and the inner surface, and the improved cleaning may limit staining or otherwise preserve the optical characteristics of the insert 74.

At least one reference marker 76 may be detected by the camera 50 for locating the region of the image associated with the imaging feature of the insert 74, and for color-correcting for variances in lighting, flash, or other optical aberrations. On suitable implementation of the reference marker is disclosed in commonly-owned U.S. Pat. No. 9,824,441, issued Nov. 21, 2017, the entire contents of which are hereby incorporated by reference, in which a quick response (QR) code of a known red color component value (or RGB, HSV, or CMYK color schemes, etc.) is affixed with adhesive to an outer surface of the waste container 26 corresponding to a position of an upper aspect of the insert 74. Further, in a manner to be described, calibration data may be associated with the unique code of the reference marker(s) 76 to account for mechanical variances of the waste container 26. Still further, in a preferred implementation, at least two reference markers 76 may be affixed to the waste container 26 in a manner to facilitate image alignment.

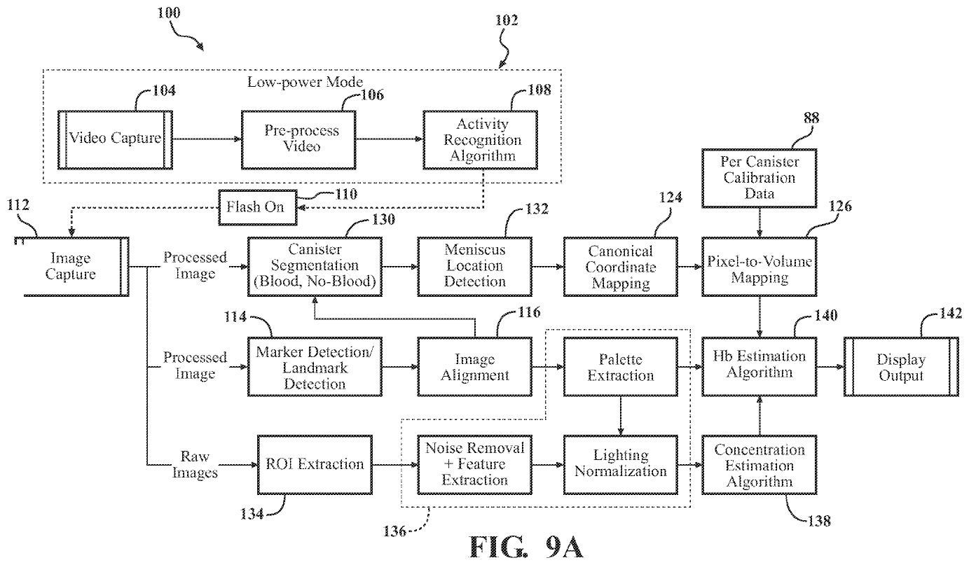

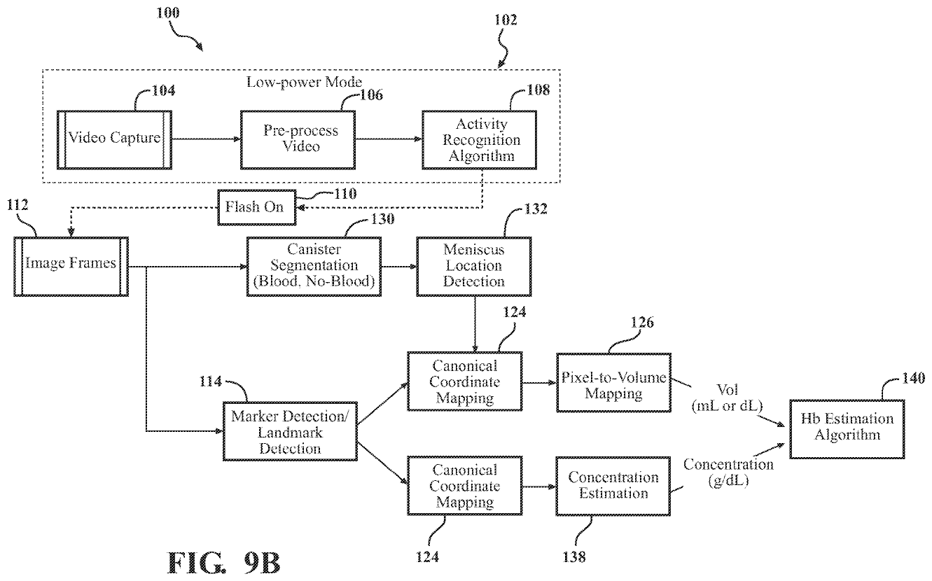

Referring now to FIGS. 9A and 9B, a method 100 of estimating the volume of blood within the waste container 26 is provided, also referred to herein as estimated or estimating blood loss. The method 100 and one or more of its steps disclosed herein are configured to be executed by the processor(s) 44 according to instructions stored on non-transitory computer readable medium. The data may be analyzed by the processor 44 on the imaging device 50, and/or the data may be transmitted for remote processing (e.g., cloud computing). The method 100 may be executed with a machine-learning (ML) model, or one or more trained neural networks may be implemented. The computer-executable instructions may be implemented using an application, applet, host, server, network, website, communication service, communication interface, hardware, firmware, software, or the like. The computer-readable medium can be stored on any suitable computer readable media such as RAM, ROM, flash memory, EEPROM, optical devices, hard drives, floppy drives, or the like.

The neural network(s) may execute algorithms trained using supervised, unsupervised, and/or semi-supervised learning methodology in order to perform the methods of the present disclosure. A diverse and representative set of training dataset is collected to train each of the neural networks on diverse scenarios both within and outside of expected uses of the system 20. Training methods may include data augmentation, pre-trained neural networks, and/or use semi-supervised learning methodology to reduce the requirement of labeled training dataset. For examples, data augmentation may increase sample size such as varying blood concentrations, semisolid, varying soiling of the sidewall of the waste container 26, lighting conditions (brightness, contrast, hue-saturation shift, PlanckianJitter, etc.), geometric transformations (rotation, homography, flips etc.), noise and blur addition, custom augmentation, hemolysis, placement of imaging device 50, clotting, detergent, suction level, or the like. Further, tolerance limit testing may also occur to determine acceptable variations for parameters such as insert depth, canister tilt, and mechanical variations of the waste container 26.

The method 100 may include operating the system in an optional standby or low-power mode (step 102). As implied by its name, the low-power mode is configured to preserve power (e.g., battery life of the imaging device 50) for periods of time in which there is no change in fluid level within the waste container 26. For example, the software of the imaging device 50 may be initiated prior to commencement of the surgical procedure, however, the medical waste collection system 20 is not drawing waste material into the waste container 26. The low-power mode 102 may include capturing, with the camera 50, the video feed (i.e., a series of image frames) (step 104) with the flash off and at a relatively lower frame rate. In one example, the frame rate is approximately 15 frames per second (fps), but other frame rates are within the scope of the present disclosure. The image frames—also referred to herein as images—are preprocessed (step 106), for example, downsampled. The downsampled images are analyzed by an activity recognition algorithm (step 108). The activity recognition algorithm detects a pixel-based location of the fluid meniscus in each of the images. The activity recognition algorithm is configured to determine whether the pixel-based location of the fluid meniscus changes by a greater amount than a predetermined threshold in a predetermined period of time. In other words, activity recognition algorithm determines whether the waste material is being drawn into the waste container 26 at a predetermined rate. In one example, the activity recognition algorithm may compare the pixel-based location the fluid meniscus between successive image frames of the video feed, and compare the changes against the predetermined threshold. If the changes remain below the predetermined threshold, the processor 44 maintains the system in the low-power mode, and foregoes executing the remainder of the method 100. If the change in the fluid meniscus is above the predetermined threshold, the processor 44 executes remaining steps of the method 100. Additionally or alternatively, the user may provide an input to the touchscreen display of the imaging device 50, a paired external device, or the like, to terminate the low-power mode and initiate an eBL mode.

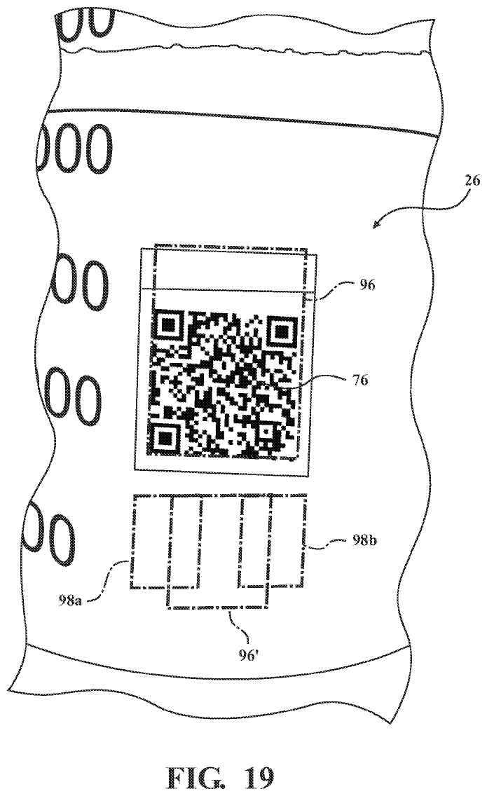

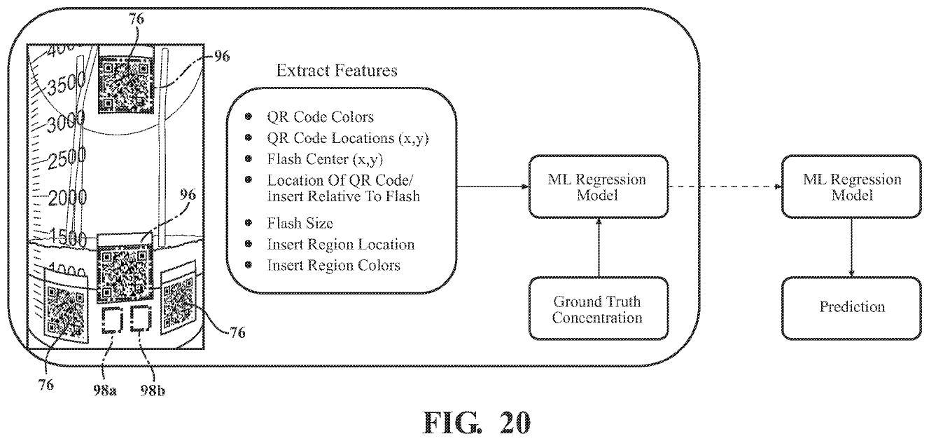

The method 100 includes activating the light source (step 110), such as activating the flash of the imaging device 50. The step is optional, and alternatively the flash of the imaging device 50 may in continuously activated. In another variant, a level of the flash of the imaging device 50 may be increased with the eBL mode. The container backlight of the waste container 26 may already be activated. Using light from both the flash and the container backlight may provide optimal illumination with minimal glare. Owing to the precise position of the camera 50 supported by the device cradle 54, it is noted that the glare may be limited to a small dot positioned above the insert 74 and the reference marker 76 (see FIGS. 19 and 20), which is a region of the image of less concern for the image analysis. Moreover, the greater signal-to-noise ratio (i.e., blood color signal to background reflection) has less effect on the image analysis, and therefore may obviate the need for a separate algorithm to reduce or remove the influence of glare. In addition to activating the flash, the feed rate of the video feed may be increased for increased data resolution.

The method 100 may include analyzing at least one of the image frames of the video feed (step 112), and preferably multiple image frames in a series according to the algorithm. In other words, every image frame may be analyzed, every other image frame, every third image frame, or the like, based on desired data resolution in view of available computing resources. In an alternative implementation not utilizing a video feed, the method 100 may include the step of capturing one or more multi-exposure photos for the subsequent analysis.

The method 100 includes the step of detecting the reference marker(s) 76 (step 114), for examples, the QR code(s), a barcode, or another marker having optically-readable data. Among other aspects to be described, the QR codes may be used to account for mechanical variances in the volume of the waste container 26. In other words, manufacturing tolerances may result in minor variations in the internal volume of the waste container 26, as well as the volumes of the insert 74 and insert mount disposed therein, may lead to inaccuracies in image-based volumetric determinations. Further, minor variations of the internal volume may occur from vacuum-induced variation in which the pressure differential from the vacuum being drawn inside the waste container 26 results in the sidewall of the waste container 26 “flexing” inwardly and displacing the waste material contained therein. Still further, additional minor variations of the internal volume may occur from thermal expansion of the waste container 26 due to body temperature fluids from the patient within the waste container 26 being at a higher temperature relative to ambient.

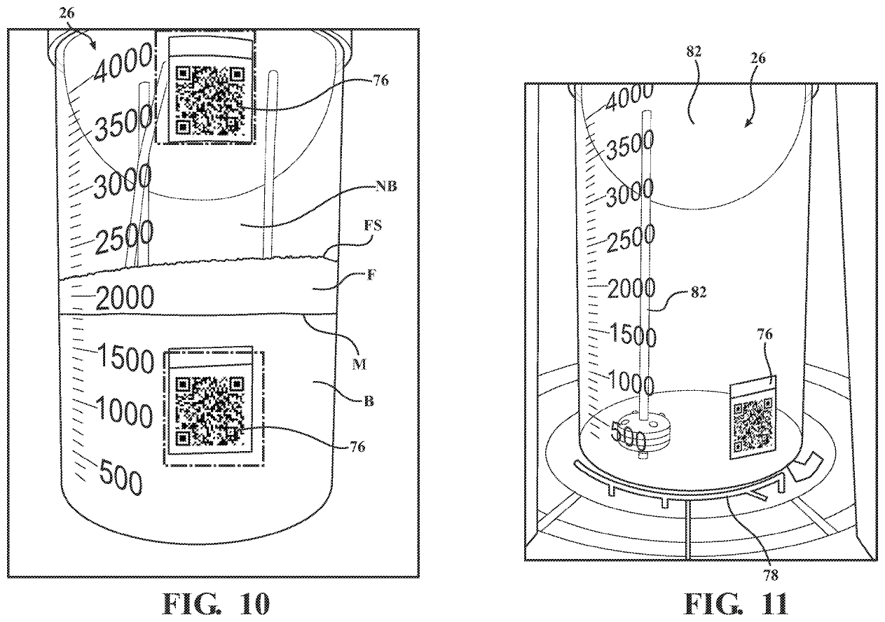

The present disclosure addresses such concerns by associating, in memory, the QR codes with a calibration-based data associated with the waste container 26. The calibration may be performed by a technician during deployment (e.g., assembly or retrofitting) of the eBL subsystem with the medical waste collection system 20. The technician may affix at least two QR codes, preferably in a vertical arrangement as shown in FIG. 10. The device cradle 54 is installed in manners previously described. The camera 50 is activated in a calibration mode, and at least one calibration run is performed in which at least one known fluid volume is suctioning into the waste container 26. The processor 44 performs the image-based volumetric determination to be described, including ascertaining a vertical, y-axis value of the fluid meniscus, and associates each of the y-axis value and the known fluid volume(s) with the unique code of the QR codes 76. The calibration data is stored on memory 88, for example, memory of the imaging device 50 or cloud-based memory. In operation, the camera 50 detects the QR codes 76, and the processor 44 accesses the calibration data from the memory 88 to perform the image-based volumetric determination as adjusted by or in view of the calibration data.

Alternative methods for container-specific calibration are contemplated. In a first example, field calibration may occur in which the calibration mode being displayed of the imaging device 50 directs the user to suction a target volume of fluid. After doing so, an input is provided to the user interface, and the processor 44 performs the image-based volumetric determination to be described, including ascertaining a vertical, y-axis value of the fluid meniscus, and associates each of the y-axis value. The user interface displays a predicted volume, and the user may provide a confirmatory input to the user interface to indicate that the predicted volume, as determined by the processor 44, is equal to the target volume of fluid. In a second example, the reference markers 76 may be affixed at precise locations and confirmed via testing, for example, in a laboratory setting. The camera 50 is activated in a calibration mode, and the y-axis position of the reference markers 76 is determined, and associated with the unique code of the reference markers 76. In a third example, the container-specific calibration data may be stored on a memory chip, such as a near-field communication (NFC) tag. The NFC tag may be detected by complementary NFC reader of the imaging device 50, and the calibration data is transmitted to the imaging device 50. The calibration data is stored in the memory 88 with the unique code of the reference markers 76. Such alternative methods may require precise placement of the reference markers 76. The step of compensating for volumetric variances of the waste container 26 is optional, and the image-based volumetric determination may be sufficiently accurate in an absence of the same.

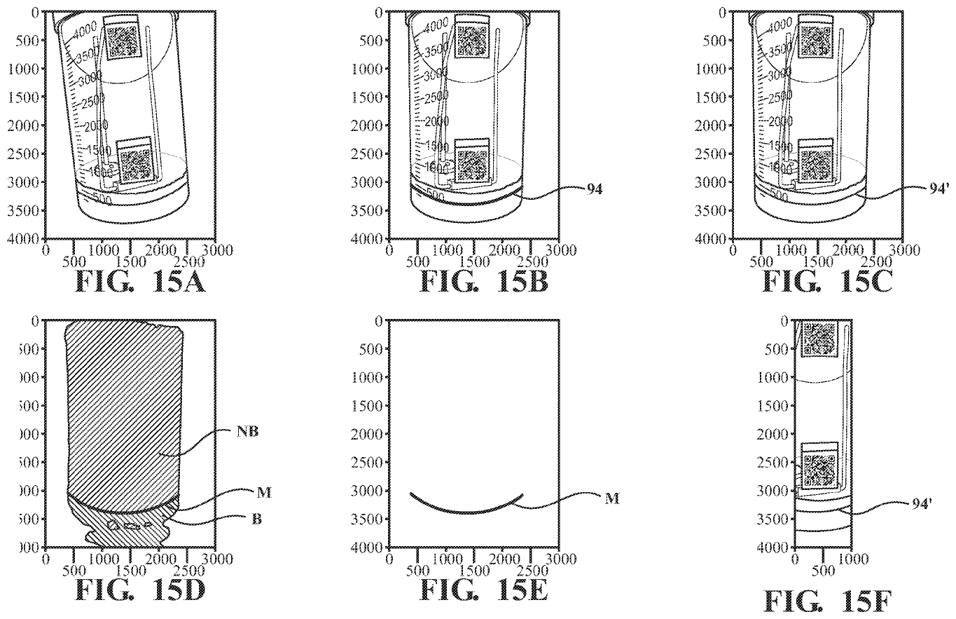

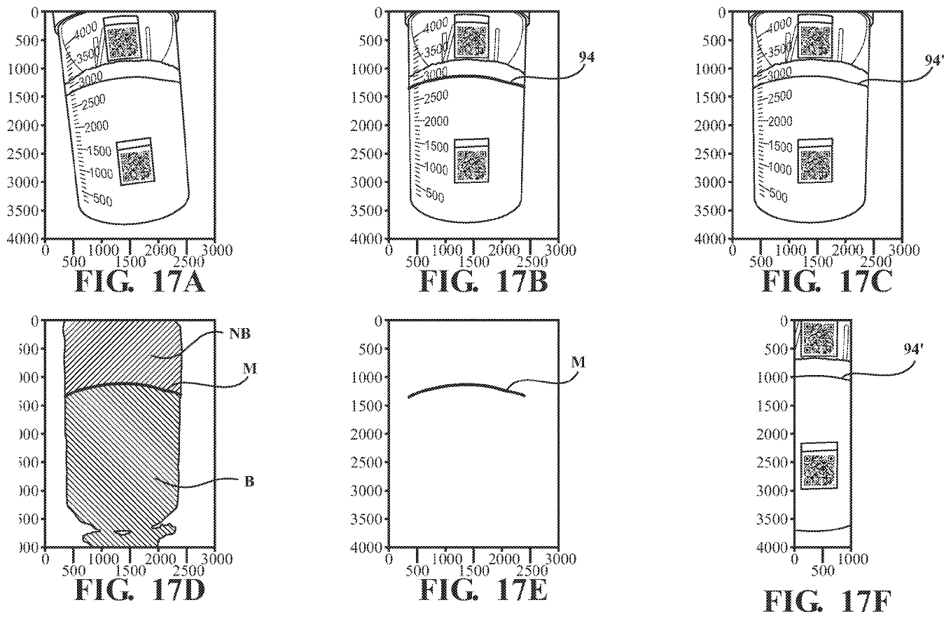

The method 100 includes the step of aligning the image (step 116). As the assessment of the fluid meniscus is on the vertical, y-axis, it is desirable for the image of the canister to be oriented vertically with sufficient precision. In other words, the step of aligning the image may account for mechanical variances in the rotational positioning of the waste container 26 within the chassis 22. The alignment may be based on features external to the waste container 26 (i.e., external alignment), features associated with or inherent to the waste container (i.e., internal alignment), or a combination thereof. With continued reference to FIG. 10, the step of aligning the image may be based on the step of detecting the reference markers 76. For example, the QR codes may be affixed to be waste container 26 with a jig or guide to be vertically arranged with sufficient vertical precision. The processor 44 is configured to detect the QR codes-as represented by the bounding boxes in FIG. 10—and rotate the image accordingly. Alignment techniques to orient and position the image within the frame in three dimensions are within the scope of the present disclosure, including homography, perspective transformation, 3D-to-2D mapping, and the like. FIGS. 15B, 16B, 17B and 18B show the effect of image rotation relative to FIGS. 15A, 16A, 17A and 18A, respectively.

Additionally or alternatively, the camera 50 may be configured to detect a landmark or fiducial of the waste container 26 or the chassis 22, and align the image accordingly. FIG. 11 represents the machine vision output in which an internal structure 78 of the chassis 22 are overlayed on the image. Additionally or alternatively, external landmarks may be disposed at fixed location(s) on the waste container 26, e.g., printed fiducials such as additional QR codes or an April tag affixed at known locations on the waste container 26. Other reference structures are contemplated, such as the sensor rod 80 of the fluid measuring subsystem, the mount of the insert 74, subcomponents of a container lid 82.

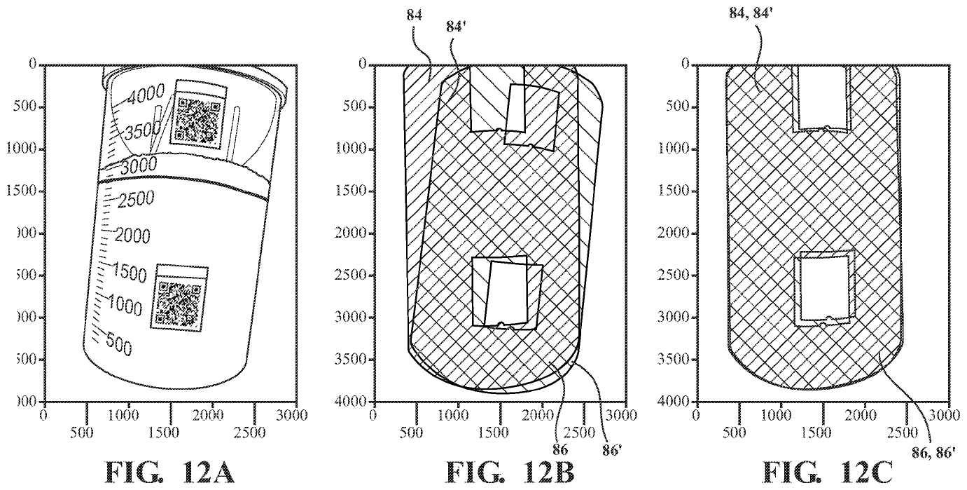

The internal alignment may include the processor 44 detecting, determining, or identifying container landmarks. With reference to FIGS. 12A-12C, the container landmarks may include a lower edge 86′, at least one side edge 84′, and/or other identifiable landmarks on the waste container 26. The edges may be identified by comparing adjacent pixels, clusters of pixels, or portions of the image. Sufficient contrast in the comparison may be indicative of a transition from internal structures of the chassis 22 to the waste container 26, that is, an edge. The lower edge 86′ may be an arcuate boundary associated with a near aspect of a base of the waste container 26. The step may implement any suitable machine vision technique and/or machine learning technique to identify the edges and/or to estimate features or dimensions of the waste container 26. The processor 44 is configured to align in the image to a predefined alignment. For example, the processor 44 may be configured to orient the side edges to be substantially vertical, represented by reference numeral 84. For another example, the processor 44 may be configured to align a lowermost aspect of the lower edge 86′ with a lower boundary of the frame of the image, represented by reference numeral 86. It is understood the external alignment may be used alone or in combination with the internal alignment.

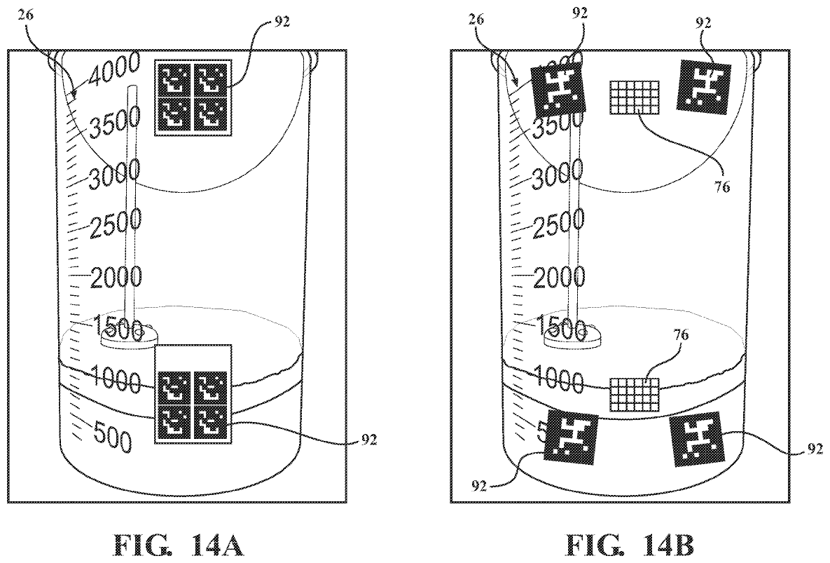

The step 116 of image alignment may also include compensating for any tilt (e.g., misalignment in fore and aft directions) of the waste container 26. The tilt may be due to a non-level floor surface, manufacturing variances, or the like. A method 118 of tilt correction is shown in FIG. 12. The method 118 includes the processor 44 receiving the image frame (step 120), and detecting reference markers 92 (step 122). With reference again to FIG. 8, the reference markers 92 are arranged to facilitate determining tilt relative to the plane of the image. For example, the markers 92 may be AruCo markers in which an alignment frame 90 is affixed and contoured to the curvature of the waste container 26, and the markers 92 arc disposed on the four sides of the frame 90. The AruCo markers provide corner localization superior to that achievable with QR codes. In one variant, the AruCo markers are not disposed on the frame 90, but rather separate AruCo affixed to the waste container 26 at predetermined locations. For example, FIGS. 14A and 14B illustrate variants in which reference markers 92 are printed with the color reference palette as well as human readable code and positioned in manners to limit unnecessary occlusion of the waste container 26 and the volumetric markings.

The image frames are segmented, and a center of mass of the waste container 26 may be determined. The method 100 may include the step of performing camera calibration, and determining a center of optical sensor. The camera calibration may be performed by determining an orientation of the imaging device 50. In an exemplary implementation in which the imaging device 50 is an iPhone 13 ProMax®, the orientation is determined based on the internal gyroscopes, and optionally from the points associated with the detection of the reference markers 92. The method 100 further includes the steps of mapping the image coordinates to canonical coordinates (step 124). The pixel point of the center of mass and the pixel point of the center of the optical sensor may each be converted to a three-dimensional point in the canonical space of the waste container 26. From the orientations of the waste container 26 and the imaging device 50, mathematical corrections are determined. For example, the mathematical corrections may be coefficients associated with the pose of the waste container 26 to minimize a least squares formulation. The corrected volume is determined (step 128) based on the image-based volume determination (step 126) and the mathematical corrections.

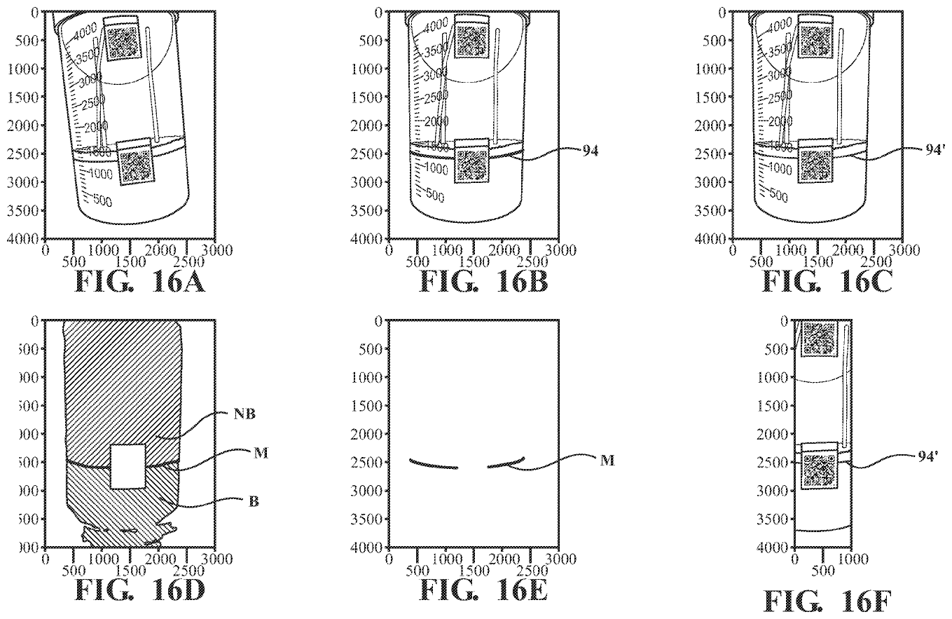

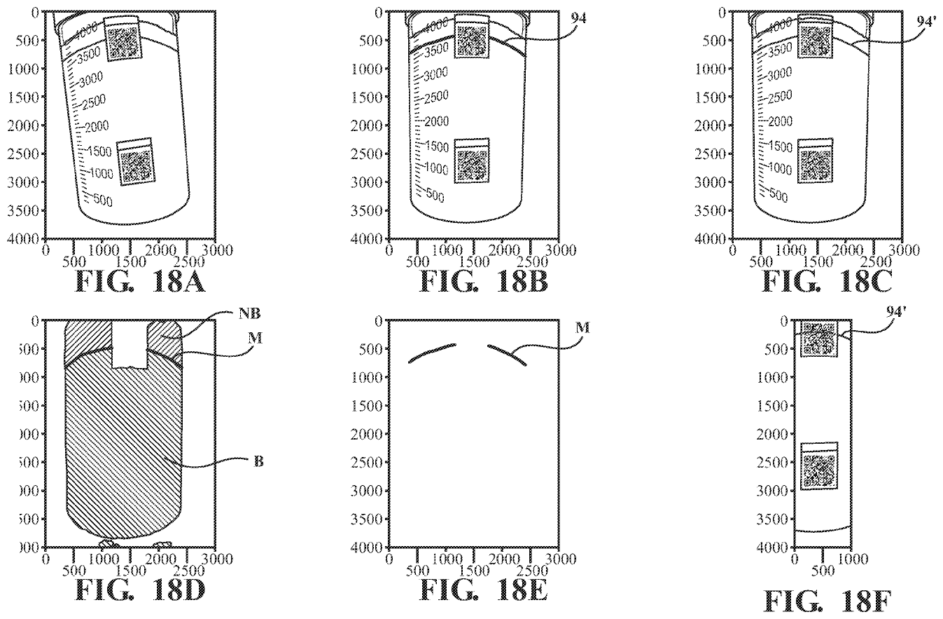

Referring again to FIGS. 9A and 9B and with further reference to FIG. 10, the method 100 includes the step of segmenting the image of the waste container 26, including the fluid contained therein. The neural network(s), hereinafter referred to in the singular, may characterize whether a pixel is of blood (B), fluid meniscus (M), or non-blood (NB). In optional variants, the neural network may further characterize whether a pixel is froth or foam (F), and/or a fluid surface (FS). In other words, each pixel is assigned a value from the neural network, and then assigned a class label based on the value. The neural network may be a deep-learning neural network such as U-Net, Mask-RCNN, DeepLab etc., or any imaged-based segmentation algorithm such as grabcut, clustering, region-growth, etc. For further examples, the neural network may use object localization, segmentation (e.g., edge detection, background subtraction, grab-cut-based algorithms, etc.), gauging, clustering, pattern recognition, template matching, feature extraction, descriptor extraction (e.g., extraction of texton maps, color histograms, HOG, SIFT, MSER (maximally stable extremal regions for removing blob-features from the selected area, etc.), feature dimensionality reduction (e.g., PCA, K-Means, linear discriminant analysis, etc.), feature selection, thresholding, positioning, color analysis, parametric regression, non-parametric regression, unsupervised or semi-supervised parametric or non-parametric regression, neural network and deep learning based methods or any other type of machine learning or machine vision. FIGS. 15D, 16D, 17D and 18D illustrate representations of a segmentation mask represented by different coloring for the non-blood, meniscus, and blood class labels. FIGS. 16D and 18D also show the reference markers 76 being excluded from the segmentation mask.

The method 100 includes determining the location of the fluid meniscus (step 132). The step 132 is based on the segmentation mask, and may include using a full width of the image to post-process segments. The aforementioned machine learning techniques may be implemented to train the segmentation network on video feeds to identify the meniscus in varying conditions, including blood concentration levels, thickening agents, lighting from the flash, lighting from the container backlight, vacuum levels of the vacuum source 30, frothiness of the waste material, cloudiness, or opaqueness of the waste container 26, and the like. Other means by which the meniscus may be detected is disclosed in commonly-owned U.S. Pat. No. 8,983,167, issued Mar. 17, 2015, the entire contents of which are hereby incorporated by reference.

The step 132 may include fitting a parabola to pixels having the class label of meniscus. FIGS. 15E, 16E, 17E and 18E illustrate representations of the fluid meniscus in which the non-blood and blood class labels are removed. In instances where the fluid meniscus is below the camera 50, the parabola includes a lower vertex and opens upwardly. Conversely, if the fluid meniscus is above the camera 50, the parabola includes a higher vertex and opens downwardly. The location of the fluid meniscus is the y-axis value of the vertex of the parabolic curve. Therefore, with the lower vertex, the location of the fluid meniscus is the minimum of the parabolic curve; and with the higher vertex, the location of the fluid meniscus is the maximum of the parabolic curve. FIGS. 15B, 16B, 17B and 18B illustrate representations of machine vision output 94 indicative of the y-axis values of the vertex overlayed on the adjusted images of the waste container 26.

The method 100 further includes the step of mapping the images to from the image coordinate space to the canonical coordinate space (step 124), as previously introduced. The step 124 may require four localization markers (e.g., two QR codes) to generate homography and map the fluid meniscus from the image coordinates to the canonical coordinates. One or more of the alignment blocks of the QR codes (e.g., top left alignment block) may be used for the transformation to two-dimensional canonical coordinates, then to three-dimensional canonical coordinates. FIGS. 15C, 16C, 17C and 18C illustrate representations of machine vision output 94′ in the canonical coordinates indicative of the y-axis values of the vertex overlayed on the adjusted images of the waste container 26, and FIGS. 15F, 16F, 17F and 18F illustrate representations of cropped adjusted images of the waste container 26 with the machine vision line 94′ overlayed. The cropped image frames in the canonical coordinate space may include the at least two reference markers, and a portion of the waste container disposed therebetween. A width of the cropped and mapped image frames is approximately equal to a width of the at least two reference markers. The cropping of the images limits the range (e.g., the width of the image) in which the meniscus may be located. The cropping of the adjusted images is optional.

The method 100 includes the step of pixel-to-volume mapping to determine the fluid volume within the waste container (step 126). The step may include retrieving or receiving the calibration data from the memory 88, for example, container-specific coefficients to account for container-specific variances, as described. The image-based volumetric determination is the y-axis value of the fluid meniscus, in the canonical coordinates, as adjusted by the calibration data. In other words, the lowest point of the meniscus, along a y-axis as defined in the image by the processor 44, may be mapped relative to a datum, and the y-axis position of the meniscus is converted to a volume in milliliters. Again, the aformentioned machine learning techniques may be implemented to train the segmentation network to convert the y-axis position of the meniscus to the fluid volume. As one example and with reference to FIG. 16C, the y-axis value of “2000” in the canonical coordinates (left axis of image) may result in a volumetric determination of approximately 1,000 mL.

The method 100 may include extracting a region of interest (ROI) from the image (step 134). With concurrent reference to FIG. 19, the processor 44 is configured to identify the reference marker 76, and process the information contained therein, to determine the corresponding location of the region of interest 96′. In other words, for example, the data within the QR code may cause the processor 44 to analyze the region of interest 96′ of the image frame at a predetermined position relative to the QR code. Further, in implementations in which the insert 74 includes the first and second imaging surfaces 75a, 75b, the reference marker 76 may contain such data to cause the processor 44 to analyze first and second regions of interest 98a, 98b, respectively.

In certain implementations, further optional steps 136 may include extracting palette colors from the reference marker(s) 76, noise removal, extracting features, and performing light normalization. The palette extraction, noise removal, and feature extraction may be performed in manner at least similar to the aformentioned U.S. Pat. No. 9,824,441. Variances in lighting may be accounted for by training the segmentation network with images in which lighting from only the flash of the camera 50 is used, light from only the container backlight is used, or combinations of both at varying levels. Among other advantages, this may permit the blood component to be determined at greater ranges of blood concentration without signal saturation. The levels of light intensity provided by the flash of the camera 50 may be of known brightness, color temperature, spectrum, and the like. Additionally or alternatively, the container backlight may provide light of a known brightness, color temperature, spectrum, and the like.

The light normalization according to known solutions may be primary used to account for variances in ambient lighting. With the camera 50 and the flash of the imaging device being supported in the device cradle 54 and configured to detect in the insert 74 disposed near a bottom of the waste container 26, the effects of directional light become relatively more pronounced. Variances in relative positioning between the camera 50 and the waste container 26 and light gradation from the flash of the imaging device 50 result in an indeterminant non-linear relationship such that empirical determinations and machine learning models may result in insufficiently accurate determinations of the concentration of the blood component within the waste material. For example, minor variations in the pose of the imaging device 50 may result in differing locations and intensities of the flash reflected on a surface of the waste container 26 as well as the qualities (e.g., color component values) of the image or image frames of the waste material. In other words, the light intensity, as detected by the camera 50, may be dependent on the location of the flash on the surface of the waste container 26. As a result, in certain implementations, the analyzing of the image or image frames to determine a concentration of the blood component may be facilitated by the neural network trained on image datascts of various positions of the camera 50 relative to the waste container 26, various positions of the flash of the imaging device 50 being reflected on a surface of the waste container 26.

Therefore, in certain implementations, the light normalization may require at least two reference markers each with a known color profile. In one example, the neural network may be trained to predict the color profile of a lower one of the reference markers 76 based on the color profiles of an upper one or more of the reference markers 76. The light normalization may include applying color correction based on the predicted color profile of the lower reference marker 76. In another example, the image or image frames to determine a concentration of the blood component may be facilitated by the neural network trained on image datasets of imaged color component values at least two, at least three, or four or more reference markers 76.

The images captured by the camera 50 may be captured under lighting of constant intensity and other characteristics. Alternatively, the step 106 is optional and may include capturing the images with the camera 50 at different levels of light intensity provided by the flash of the camera 50, wherein the processor 44 utilizes an algorithm that compensates for the levels of light intensity. For example, a first image is captured under a high flash intensity, a second image is captured under medium flash intensity, a third image is captured under low flash intensity, and so on, allowing for increased dynamic range of the image captured and associated increased range of blood component measurable by the algorithm. Another light normalization approach includes intensity gradient normalization in which the distance and/or angle of the flash is accounted for.

The method 100 includes the step 138 may further include analyzing the region of the image associated with the imaging feature of the insert 74 to quantify a concentration the blood component in the waste material. The analysis may be carried out in a manner disclosed in the aforementioned U.S. Pat. No. 8,792,693 in which a parametric model or a template matching algorithm is executed to determine the concentration of the blood component associated with fluid within the waste container 26. In particular, the processor 44 may be configured to extract a color component value (e.g., a redness value) from the image, and execute the trained algorithm to determine the concentration of the blood component on a pixel-by-pixel or other suitable basis. The hemoglobin (Hb) or blood loss (eBL) is estimated with a hemoglobin estimation algorithm (step 140) and based on the image-based volumetric determination and the concentration of the blood component. The step 140 may be a product of the two, or based on another suitable computation, such as Equation 1.

$$δ M = VC ( \frac{δ L}{L} ) + V δ {C}_{a l g o} + C δ {V}_{a l g o} \qquad ( 1 )$$

FIG. 20 shows a variant of the method 100 in the machine-learning environment in which certain features arc extracted using the aligned image and location of the reference markers 76, and light normalization is performed in the color space. The variant of FIG. 9B includes certain steps being consolidated to be performed by the neural network analyzing the image frames of the video feed. In particular, by training the neural network on a diverse and representative set of training dataset, the steps of region of interest extraction, color palette extraction, noise removal, feature extraction, and lighting normalization may not be discrete steps performed by the method 100, but otherwise carried out by the neural network without loss of accuracy of the hemoglobin estimations.

In another variant, the method 100 depicted in FIG. 9A may be implemented in boundary cases to be described, and the method 100 depicted in FIG. 9B may be otherwise implemented by default. The boundary cases may be instances in which the volume of the waste material is below a predetermined volume. The predetermined volume may be based on the fluid level of the waste material not being above the insert 74 disposed within the waste container 26. For example, the predetermined volume may be less than approximately 1,000 mL, which may include 800 mL of prefill liquid and an additional 400 mL of waste material collected during the procedure. Below this predetermined volume, the method 100 of FIG. 9A (and FIG. 20) implemented by the ML model may be better suited for these threshold cases. Otherwise, with fluid volumes that are above the predetermined volume, the method 100 of FIG. 9B implemented by the trained neural network may be more accurate at a wider range of blood concentrations. The processor 44 may be configured to implement either variant of the method 100 based on the fluid volume or other considerations or determinations.

The estimated blood loss may be displayed on one or more displays (step 142), for example, the touchscreen display of the imaging device 50. Additionally or alternatively, the eBL may be wirelessly transmitted to the user interface 52 of the medical waste collection system 20 or another display terminal within the surgical suite. As should be readily appreciated from the foregoing disclosure, the eBL may be displayed and updated at desired intervals or in real-time. In particular, in the video mode the camera 50 may take the video feed in which each of the blood component and the fluid volume is repeatedly determined in a near-instantaneous manner. Moreover, the touchscreen display of the camera 50 shows the field of view of the camera, and therefore the ability to visualize the internal volume the waste container 26 is generally unimpeded, and further may be augmented with information of use to the user.

It is contemplated that, in certain implementations, the camera 50 may be paired in bidirectional wireless communication with the medical waste collection system 20, and more particularly the controller 42. In such an arrangement, the fluid measuring subsystem may be utilized as an alternative or in combination with the image-based volumetric determinations. The data from the fluid measuring subsystem may also be leveraged to refine the machine learning. Alerts may be provided via the user interface 52 should the blood loss of the patient exceed predetermined or selected limits. The blood loss data associated with the medical procedure may be transmitted to an electronic medical record.

Additional alerts or guardrails are contemplated by the present disclosure. In certain implementations, the camera 50 and processor 44 may be configured to determine and provide an event detector as to a state of the waste container 26. In other words, the machine learning algorithm could be extended to determine whether suction is on or off, whether the waste material is flowing into the waste container 26, whether the waste container 26 is static or emptying, or the like. Likewise, the machine learning algorithm could be extended to determine whether a clot or other debris has become lodged near or in front of the imaging feature of the insert 74, and/or whether the insert 74 is missing, dislodged, or otherwise improperly positioned beyond a predetermined threshold. While decreases in the concentration of the blood component are permissible an error alert may be provided if the processor 44 determines the eBL has decreased.

Additionally or alternatively, a reference image of the insert 74 may be used to determine the blood component, e.g., a first image prior to the procedure in which there is no blood in the container. The reference image may be compared against some of all subsequent images. Utilizing the reference image provides, for example, for compensating for changes in a color of the imaging feature of the insert 74 as well as for variations in light intensity over multiple uses inside the waste container 26 that is subjected to repeated dirtying and cleaning cycles. It is noted that blood concentration ratios based on the images to the reference image may not directly depend on the light intensity.

In certain implementations, the machine learning algorithm could be extended to determine an extent of haze on the inner or outer surface of the waste container 26, damage (e.g., scratches) to the outer surface of the waste container 26, soiling of the lens of the camera 50, or the like. For example, by capturing the images, such as the reference image, prior to and during each procedure, a rate of dirtying of the waste container 26 or the insert 74 may be determined. For example, it may be assumed that absent dirtying, analysis of the reference images should be within a predetermined similarly threshold. If the analysis of the reference images exceeds the predetermined similarly threshold, it may be considered that the waste container 26 is too solid for reliable analysis beyond a confidence interval. Corresponding predicative alerts or corrective actions may be provided. In particular, based on the analysis, predictive maintenance may be summoned in which a technician cleans the inner and outer surfaces of the waste container 26, and replaces the insert 74.

Further, the machine learning algorithm may be extended to determine the heterogeneity of the waste material within the waste container 26. For example, by analyzing the imaging region containing fluid as determined by the segmentation network. Based on computing feature of heterogeneity such as edge score, standard deviations of pixel values, HOG, or other similar feature, the user maybe warned for scenarios when the fluidic content is not homogenous as such situations may lead to inaccurate analysis of the blood content within the fluid. The user may in such situation be directed to agitate the fluid, e.g., by mechanical motion or by suction of water at high suction pressure, thereby properly mixing the fluidic content within the imaging container for accurate analysis of the blood content. Once a correct mixing has been achieved and the heterogeneity score is below a certain threshold, the analysis of blood content can proceed normally.

In another implementation, the camera 50 may be integrated on the chassis 22, and not necessarily the mobile device supported on the device cradle 54. In such an implementation, the camera 50 may be at least one digital camera coupled to the chassis 22 in any suitable location, such as within the front casing 46. Additionally or alternatively, the digital camera may be coupled to the waste container 26 to be positioned internal and/or external to the waste volume. For example, the digital camera may be coupled to the container lid 82 and oriented downwardly. Multiple cameras may be utilized in combination from which the images are analyzed with the analysis synthetized by the machine learning algorithm for improved accuracy and redundancy.

Several implementations have been discussed in the foregoing description. However, the implementations discussed herein are not intended to be exhaustive or limit the invention to any particular form. Modifications and variations are possible in light of the above teachings and may be practiced otherwise than as specifically described. For example, the methods disclosed herein may be performed on a waste container that is not disposed on a mobile chassis. In other words, a device cradle-such as that disclosed in commonly-owned U.S. Pat. No. 10,641,644, issued May 5, 2020, the entire contents of which are hereby incorporated by reference, may be provided in which a freestanding canister is situated. For example, the blood component may be hemoglobin, or otherwise may be one or more of whole blood, red blood cells, platelets, plasma, white blood cells, analytes, and the like. The methods may also be used to estimate a concentration and an amount of a non-blood component within the waste container 26, such as saline, ascites, bile, irrigating fluids, saliva, gastric fluid, mucus, pleural fluid, interstitial fluid, urine, fecal matter, or the like. The medical waste collection system 20 may communicate with other systems to form a fluid management ecosystem for generating a substantially comprehensive estimate of extracorporeal blood volume, total blood loss, patient euvolemia status, or the like. Moreover, certain inventive aspects of the present disclosure are made with reference to the following exemplary clauses:

Clause 1-A method of estimating blood loss of waste material within a waste container of a medical waste collection system in which an imaging device is supported in a device cradle, the method comprising the steps of: capturing, with the imaging device, an image of the waste container and the waste material disposed therein; analyzing, with one or more processors, the images to determine the volume of the waste material within the waste container; analyzing, with one or more processors, the images to determine a concentration of the blood component within the waste material; estimating, with the one or more processors, the blood loss based on the determined volume and the determined concentration of the blood component; and displaying, on a display, the estimated blood loss.

Clause 2-A method of estimating blood loss of waste material within a waste container of a medical waste collection system in which an imaging device is supported in a device cradle, the method comprising the steps of: capturing, with the imaging device, a video feed of the waste container and the waste material disposed therein as a vacuum source of the medical waste collection system is drawing the waste material into the waste container; segmenting, with one or more processors, image frames of the video feed by characterizing whether pixel values of each of the image frames are blood, meniscus, or non-blood, and optionally, froth or a fluid surface; assigning, with the one or more processors, class labels based on the pixel values; determining, with the one or more processors, a location of the fluid meniscus based on the class label of meniscus; mapping the image frames from an image coordinate space to a canonical coordinate space; and determining, with the one or more processors, a volume of the waste material within the waste container based on the location of the fluid meniscus.

Clause 3—The method of any one of clause 2, further comprising: detecting, with one or more processors, at least two reference markers affixed to the waste container; and cropping the mapped image frames in the canonical coordinate space to include the at least two reference markers, and a portion of the waste container disposed therebetween.

Clause 4—The method of clause 3, further comprising aligning, with one or more processors, the images based on relative positioning of the at least two reference markers.

Clause 5-A device cradle for supporting an imaging device for capturing images of a waste container of a medical waste collection system, wherein a front casing of the medical waste collection system defines a window, the device cradle comprising: a shield coupled to the front casing, wherein the shield defines a rear recess configured to cover the window, a front recess, and an aperture providing fluid communication between the rear recess and the front recess, wherein the front recess is sized to receive the imaging device and the aperture positioned relative to the front recess to be aligned with a camera and a flash of the imaging device.

Clause 6—The device cradle of clause 5, further comprising: at least one arm coupling the shield to the front casing; and a hinge coupling the at least one arm and the shield and configured to permit the shield, with the imaging device disposed therein, to be pivoted to a configuration in which the window is viewable.

Clause 7—The device cradle of clause 6, further comprising a front housing movably coupled to the shield.

Clause 8—The device cradle of any one of clauses 5-7, wherein at least nearly an entirety of the waste container is con figured to be viewable in a field of view of the camera of the imaging device.

1. A method of estimating blood loss of waste material within a waste container of a medical waste collection system in which an imaging device is supported in a device cradle, the method comprising the steps of:
capturing, with the imaging device, a video feed of the waste container and the waste material disposed therein as a vacuum source of the medical waste collection system is drawing the waste material into the waste container;
analyzing, with one or more processors, the image frames of the video feed to determine a volume of the waste material within the waste container;
analyzing, with one or more processors, the image frames to determine a concentration of a blood component within the waste material;
estimating, with the one or more processors, the blood loss based on the determined volume and the determined concentration of the blood component; and
displaying, on a display, the estimated blood loss.

2-7. (canceled)

8. The method of claim 1, further comprising:
capturing, with the imaging device, the video feed at a first frame rate;
determining, with the one or more processors, a rate at which the volume of the waste material is increasing within the waste container; and
capturing, with the imaging device, the video feed at a second frame rate greater than the first frame rate if the rate greater than a predetermined threshold.

9. The method of claim 8, further comprising activating or increasing a level of a flash of the imaging device if the rate is greater than the predetermined threshold.

10. The method of claim 9, further comprising:
detecting, with the one or more processors, a pixel-based location of a fluid meniscus in the image frames; and
determining, with the one or more processors, whether the pixel-based location of the fluid meniscus changes by an amount greater than a predetermined threshold in a predetermined period of time.

11. The method of claim 10, further comprising down sampling the image frames prior to the step of detecting the pixel-based location of the fluid meniscus.

12. The method of claim 1, further comprising:
detecting, with one or more processors, at least two reference markers affixed to the waste container; and
aligning, with one or more processors, the image frames based on relative positioning of the at least two reference markers.

13. The method of claim 12, wherein the at least two reference markers are quick response (QR) codes arranged in a generally vertical configuration, wherein the step of aligning the image frames further comprises aligning the QR codes to be exactly vertical.

14. The method of claim 1, further comprising:
segmenting, with the one or more processors, the image frames by characterizing whether pixel values of each of the image frames are blood, meniscus, or non-blood, and optionally, froth or a fluid surface; and
assigning class labels based on the pixel values.

15. The method of claim 14, further comprising determining a location of the fluid meniscus based on the class label of meniscus.

16. The method of claim 1, further comprising mapping the image frames from an image coordinate space to a canonical coordinate space by generating homography of the least two localization markers, and transforming the image coordinates to two-dimensional canonical coordinates.

17. (canceled)

18. The method of claim 16, further comprising cropping the mapped image frames in the canonical coordinate space to include the at least two reference markers, and a portion of the waste container disposed therebetween.

19. The method of claim 16, wherein the step of analyzing the image frames to determine the volume of the waste material further comprises:
receiving container-specific calibration data; and
converting a y-axis value of the mapped image frames in the canonical coordinate space to a volumetric value.

20. A method of estimating blood loss of waste material within a waste container of a medical waste collection system in which an imaging device is supported in a device cradle, the method comprising the steps of:
capturing, with the imaging device, a video feed of the waste container and waste material disposed therein; and
processing, with one or more processors, image frames of the video feed, wherein the step of processing the image frames further comprises:
determining a location of a fluid meniscus;
aligning the image frames;
mapping the image frames from an image coordinate space to a canonical coordinate space; and
converting a y-axis value of the mapped image in the canonical coordinate to a determined volume;
detecting, with one or more processors, at least two reference markers affixed to the waste container, wherein the reference marker includes location data; and
extracting, with one or more processors, a region of interest in a raw image based on the location data;
analyzing, with one or more processors, the image to determine a concentration of a blood component within the waste material;
estimating, with the one or more processors, the blood loss based on the determined volume and the determined concentration of the blood component; and
displaying, on a display, the estimated blood loss.

21. The method of claim 20, wherein the step of determining the location of a fluid meniscus further comprises:
segmenting, with the one or more processors, the image frames by characterizing whether pixel values of each of the image frames are blood, meniscus, or non-blood; and
assigning, with one or more processors, class labels based on the pixel values, wherein the location of the fluid meniscus based on the class label of meniscus.

22. The method of claim 20, wherein the step of aligning the image further comprises detecting and aligning at least two reference markers to be exactly vertical.

23. The method of claim 20, wherein the step of mapping the image further comprises:
generating, with one or more processors, homography of the at least two localization markers; and
transforming, with one or more processors, the image coordinates to two-dimensional canonical coordinates.

24. The method of claim 20, further comprising cropping the mapped image in the canonical coordinate space to include the at least two reference markers, and a portion of the waste container disposed therebetween.

25. The method of claim 20, wherein the step of analyzing the image frames to determine the volume of the waste material further comprises applying container-specific calibration data.

26-31. (canceled)

32. A method of estimating blood loss of waste material within a waste container of a medical waste collection system in which an imaging device is supported in a device cradle, the method comprising the steps of:
capturing, with the imaging device, a video feed of the waste container and the waste material disposed therein as a vacuum source of the medical waste collection system is drawing the waste material into the waste container;
analyzing, with one or more processors, image frames of the video feed to determine a volume of the waste material within the waste container;
analyzing, with one or more processors, the image frames of the video feed to determine a concentration of a blood component within the waste material, wherein the step of analyzing the image frames is facilitated by a neural network trained on image datasets of at least one of relative positioning of a camera of the imaging device relative to the waste container, position of a flash reflected on a surface of the waste container, color component values associated with at least two reference markers affixed to the surface of the waste container, and color component values associated with an imaging surface of an insert disposed within the waste container;
estimating, with the one or more processors, the blood loss based on the volume and the determined concentration of the blood component; and
displaying, on a display, the estimated blood loss.

33. The method of claim 32, wherein the step of analyzing the image frames is facilitated by the neural network being further trained on the image datasets of regions of interest associated with at least two imaging surfaces of the insert with each of the at least two imaging surfaces providing a different color component value to the waste material disposed between the insert and the waste container.

34. The method of claim 32, wherein the step of analyzing the image frames is facilitated by the neural network being further trained on the image datasets of a region of interest associated with the imaging surface of the insert providing a color gradient to the waste material disposed between the insert and the waste container.

35. The method of claim 32, wherein the step of analyzing the image frames is facilitated by the neural network further trained on the image datasets of at least one of various volumes of the waste material and differing phase characteristics of the waste material.

36. The method of claim 32, further comprising:
mapping the image frames from an image coordinate space to a canonical coordinate space by generating homography of the at least two localization markers, and transforming the image coordinates to two-dimensional canonical coordinates; and
providing the image frames in the canonical coordinate space to the neural network.

37. The method of claim 36, further comprising cropping the mapped image frames in the canonical coordinate space to include the at least two reference markers, and a portion of the waste container disposed therebetween.

38. The method of claim 36, wherein the step of analyzing the image frames to determine the volume of the waste material further comprises:
receiving container-specific calibration data; and
converting a y-axis value of the mapped image frames in the canonical coordinate to a volumetric value.
